(19) United States
(12) Patent Application Publication
(10) Pub. No.: US 2020/0350041 A1
(43) Pub. Date: Nov. 5, 2020

(54) METHODS OF FORECASTING ENROLLMENT RATE IN CLINICAL TRIAL

(71) Applicant: Gen LI, East Lyme, CT (US)

(72) Inventor: Gen LI, East Lyme, CT (US)

(21) Appl. No.: 16/931,692

(22) Filed: Jul. 17, 2020

Related U.S. Application Data
(63) Continuation of application No. 16/124,369, filed on Sep. 7, 2018.
Application No. 16/124,369 is a continuation of application No. 14/818,438, filed on Aug. 5, 2015, now abandoned.
(60) Provisional application No. 62/033,844, filed on Aug. 6, 2014.

(51) Int. Cl. G16H 10/20; G16H 40/20

(52) U.S. Cl. CPC G16H 10/20; G16H 40/20

(57) ABSTRACT

In one embodiment, the present invention provides a method of designing a clinical trial enrollment plan, comprising the use of non-linear regression analysis to model the relationship between the number of investigator sites and the site enrollment rates, or the relationship between the number of investigator sites and the trial enrollment rates. One or more parameters such as the number of investigator sites, site enrollment rates, and/or trial enrollment rates can then be extrapolated from said regression analysis, wherein said extrapolated parameters are used in the design of one or more clinical trial enrollment plans

### CROSS-REFERENCE TO RELATED APPLICATIONS

This application claims the benefit of priority of U.S. Ser. No. 62/033,844, filed Aug. 6, 2014. The entire content and disclosure of the preceding application is incorporated by reference into this application.

### FIELD OF THE INVENTION

This invention relates generally to methods of improving operational effectiveness in clinical trial planning and execution.

### BACKGROUND OF THE INVENTION

To bring new medicines to needy patients faster is a perennial challenge to clinical development organizations around the world. Longer enrollment cycle time, raising development costs, and declining output are some of the challenges in conducting clinical trials. There are limited understanding on the root causes and true drivers behind these struggles.

In general, there are different factors impacting enrollment cycle times. A specifically defined patient population for a particular disease, for example, can impact the ability of trial sites to identify and recruit patients in a defined period of time, thereby impacting the enrollment cycle time. An experienced and successful investigator/site may have better ability to enroll qualified patients compared to an inexperienced investigator/site. Higher portion of experience sites in a pool of sites deployed by a clinical trial can result in shorter enrollment cycle time.

Instinctively, when there are more sites/investigators being deployed for a trial with a defined number of patients needed, one would expect shortened enrollment cycle time. As clinical development organizations are under pressure to deliver new product faster, senior management often is happy to put seemingly “unlimited” resources behind pivotal clinical trials to evaluate promising drug candidates. A simple logic is to add more sites to the pool for enrollment, aiming to “proportionately” shorten enrollment cycle time. However, the goal of proportionally shorten enrollment cycle time is rarely achieved

In another common scenario, when transitioning to a Phase III trial after a successful Phase II trial, people often “extrapolate” the operational results from the Phase II trials(s) to the Phase III trial(s). Using the enrollment rates from the Phase II trial(s) to calculate the number of sites needed for the Phase III trials, one hope to achieve similar enrollment cycle time as what happened in Phase II trial(s). However, the eventual enrollment cycle time is unlikely to be close to the calculation; instead, the enrollment cycle times are generally substantially longer in this situation.

It has been reported that adding extra sites to a clinical trial has only limited impact to enrollment cycle time (1). In order to shorten enrollment cycle time and minimize costs, one usually plan for an optimized number of sites for a clinical trial. However, it is not clear whether there is a pattern between the number of site deployed in a clinical trial and enrollment cycle time, and whether it is possible to define such pattern in a simple and universally applicable mathematical relationship.

Thus, there is a need to develop new methodology that would enable one to quantitatively identify and realize opportunities for improvement in clinical trial execution.

### SUMMARY OF THE INVENTION

This invention provides a method of improving operational effectiveness in planning and executing clinical trial. It reveals and establishes patterns and mathematical relationships between clinical trial enrollment rate (CTER) and the number of investigator sites (N), and/or between gross site enrollment rate (GSER) and the number of investigator sites (N). In one embodiment, this invention can define boundaries for clinical trial operational deliverables, and make it easier to identify specific opportunities to improve operational deliverables, such as reducing number of sites needed, and/or shorten enrollment cycle times.

Upon collecting operations data from a number of clinical trials for a disease condition, such as number of investigator sites, gross site enrollment rate (GSER, i.e. number of patients per site per unit period of time), and/or clinical trial enrollment rate (CTER, i.e. number of patients enrolled in a defined unit period of time), the relationship between the number of investigator sites and the site enrollment rates, or the relationship between the number of investigator sites and the trial enrollment rates can be modeled by non-linear regression analysis. In one embodiment, a graph of investigator sites vs site enrollment rates, or a graph of investigator sites vs trial enrollment rates can be plotted, and the resulting graphs or charts depict relationships between CTER and the number of investigator sites, or between GSER and the number of investigator sites. As vast majority of data points in these charts fall in a narrow band, and in definable pattern, it is feasible to use these graphs to forecast enrollment rate and other operational deliverables. In one embodiment, one can extrapolate from such graphs the correspondence between the number of investigator sites and site enrollment rate. In another embodiment, one can extrapolate from such graphs the correspondence between the number of investigator sites and trial enrollment rate.

In one embodiment, the present invention provides a method of designing a clinical trial enrollment plan, comprising the steps of: (i) obtaining clinical trial parameters from a plurality of historical clinical trials for a disease condition, said clinical trial parameters comprise the number of investigator sites, site enrollment rates, and trial enrollment rates, wherein the site enrollment rate is defined as the number of patients enrolled at a single investigator site in a unit of time, and the trial enrollment rate is defined as the number of patients enrolled in a unit of time; (ii) conducting non-linear regression analysis to model the relationship between the number of investigator sites and the site enrollment rates, or the relationship between the number of investigator sites and the trial enrollment rates; and (iii) extrapolating from said regression analysis one or more parameters selected from the group consisting of the number of investigator sites, site enrollment rates, and trial enrollment rates, wherein said extrapolated parameters are used in the design of one or more clinical trial enrollment plans.

In one embodiment, for mathematical simplicity, one can group site enrollment rate data into a set of data bins. The original individual clinical trial site (N) and site enrollment rate (GSER) data value falling in a given small interval, a bin, are then replaced by a representative value, a median clinical trial site (N), and a median site enrollment rate (GSER) in each data bin. Subsequently, a graph of median investigator sites vs median site enrollment rates is plotted.

In another embodiment, one can group trial enrollment rate data into a set of data bins. The original individual clinical trial site (N) and trial enrollment rate (CTER) data value falling in a given small interval, a bin, are then replaced by a representative value, a median clinical trial site (N), and a median clinical trial enrollment rate (CTER) in each data bin. Subsequently, a graph of median investigator sites vs median trial enrollment rates is plotted.

### BRIEF DESCRIPTION OF THE DRAWINGS

FIG. 1 shows a chart of Number of Sites (N) vs Clinical Trial Enrollment Rate (CTER) for clinical trials of a single metabolic disease condition with binned data.

FIG. 2 shows a chart of Number of Sites (N) vs Clinical Trial Enrollment Rate (CTER) for clinical trials of a single respiratory disease condition with binned data.

FIG. 3 shows a chart of Number of Sites (N) vs Clinical Trial Enrollment Rate (CTER) for clinical trials of a single neurologic disease condition with binned data.

FIG. 4 shows the graph of FIG. 1 fitted by the mathematical formula CTER disclosed herein with binned data.

FIG. 5 shows the graph of FIG. 2 fitted by the mathematical formula CTER disclosed herein with binned data.

FIG. 6 shows the graph of FIG. 3 fitted by the mathematical formula CTER disclosed herein with binned data.

FIG. 7 shows a chart of Number of Sites (N) vs Gross Site Enrollment Rate (GSER) for clinical trials of a single disease condition with binned data.

FIG. 8 shows a chart of Number of Sites (N) vs Gross Site Enrollment Rate (GSER) for clinical trials of a single respiratory disease condition (left panel), and clinical trials of a single neurologic disease condition (right panel) with binned data.

FIG. 9 shows the graph of FIG. 7 fitted by the mathematical formula GSER disclosed herein with binned data.

FIG. 10 shows the graph of FIG. 8, left panel, fitted by the mathematical formula GSER disclosed herein with binned data.

FIG. 11 shows the graph of FIG. 8, right panel, fitted by the mathematical formula GSER disclosed herein with binned data.

FIG. 12 shows a chart of Number of Sites (N) vs Gross Site Enrollment Rate (GSER) for clinical trials of a single disease condition with original data (not binned).

### DETAILED DESCRIPTION OF THE INVENTION

The present invention is a part of an integrated effort to build a conceptual structure for managing study operations in clinical development by focusing on forecasting enrollment rate at clinical trial level (Clinical Trial Enrollment Rate, CTER) and site level (Gross Site Enrollment Rate, GSER).

As used herein, Clinical Trial Enrollment Rate (CTER) refers to the number of patients enrolled in a defined unit period of time, e.g. a month, in the duration of clinical trial enrollment period.

As used herein, Gross Site Enrollment Rate (GSER) refers to the number of patients enrolled by a single site in a defined unit period of time, e.g. a month, in the duration of clinical trial enrollment period.

In one embodiment, the present invention determines and establishes mathematical relationships between the concept of CTER and number of investigator sites (N), or the concept of GSER and number of investigator sites. They are integrated components of a comprehensive conceptual framework.

In one embodiment, the present invention employs non-linear regression analysis. Not all clinical trials will and can be fitted to the equations disclosed herein perfectly. However, even though most clinical trials will not have a perfect fit, the present invention shows that the “imperfect fit” is one of the most important value proposition of the mathematical models disclosed herein. It is predicted that the following are factors that will cause “imperfect fit”:
A targeted age group too far away from “median” age group;
One or more biochemical and/or physiological and/or genetic measure(s) too far away from the “median” measures;
Targeted disease status too far away from a “regular” patient population;
Any other inclusion/exclusion criteria making the clinical trial too “unique”.

The above list is not an inclusive list; nevertheless, the database of the present invention is comprehensive enough to explain, often quantitatively, vast majority of the impact from these factors.

Establishing the relationship between number of investigator sites and trial level enrollment rate, as well as the relationship between number of investigator sites and site level enrollment rate, are significant addition to the currently more or less empty toolkit available to those in the planning and execution of clinical trials.

To be able to define an operational boundary is a leap forward to improve planning of clinical trials. The sets of mathematical relationships and graphs disclosed herein, however, can do more than that. For a planned clinical trial, when one have the targeted number of enrollment defined, and desired enrollment cycle time are set, one can use the mathematical equations and/or graphs to optimize the number of sites needed for the trial.

In one embodiment, upon collecting the data and plotting a graph as discussed herein, one or ordinary skill in the art would readily perform curve fitting to fit the graph with one or more mathematical formula. As it is well-known in the art, curve fitting is the process of constructing a curve or mathematical function that has the best fit to a series of data points. For example, curve fitting can involve either interpolation, where an exact fit to the data is required, or smoothing in which a “smooth” function is constructed that approximately fits the data. Fitted curves can be used as an aid for data visualization, to infer values of a function where no data are available, and to summarize the relationships among two or more variables. There are a number of commercially available statistical packages or software for curve fitting.

In one embodiment, the present invention provides a method of improving operational effectiveness in a clinical trial, comprising the steps of: (i) obtaining clinical trial parameters from a plurality of historical clinical trials for a disease condition, said clinical trial parameters comprise investigator sites and site enrollment rates, wherein said site enrollment rate is defined as the number of patients enrolled at a single investigator site in a unit of time; (ii) plotting a graph of investigator sites vs site enrollment rates; and (iii) extrapolating from said graph a number of investigator sites corresponding to a desired site enrollment rate, or a number of site enrollment rate corresponding to a desired number of investigator sites, wherein said extrapolated number of investigator sites or number of site enrollment rate would provide information for improving operational effectiveness of a clinical trial. In one embodiment, the investigator sites and site enrollment rates are further converted into median investigator sites and median site enrollment rates before plotting said graph.

In one embodiment, the unit of time is one month. In one embodiment, the disease condition is a metabolic disease condition, a respiratory disease condition, or a neurologic disease condition, and other disease conditions studied by randomized clinical trials.

In one embodiment, the graph of investigator sites vs site enrollment rate can be fitted by the following formula:

GSER=a*e^{bN}+c

wherein GSER is Gross Site Enrollment Rate; a, b, and c are parameters for a set of clinical trials of a disease condition; b is a negative constant for a set of clinical trials; and N is investigator sites. In one embodiment, the lower limit of site level enrollment rate is c.

In one embodiment, the GSER is related to Site Effectiveness Index (SEI) and Average Site Enrollment Rate (ASER) as: GSER=SEI×ASER, wherein

$$SEI = \frac{{\int }_{i = 1}^{N} ( {Et}_{i} - {St}_{i} )}{( {Et}_{s} - {St}_{s} ) \times N} ,$$

Eti is the date site i closed for patient enrollment; Sti is the date site i opened for patient enrollment; N is maximum number of sites opened for enrollment in the duration of patient enrollment at the study level; Ets is the date clinical trial closed for patient enrollment; and Sts is the date clinical trial opened for patient enrollment; wherein

$$ASER = \frac{TE}{{\int }_{i = 1}^{N} ( E {t}_{i} - {St}_{i} )} ,$$

### TE is Total Enrollment.

In another embodiment, the present invention provides a method of improving operational effectiveness of a clinical trial, comprising the steps of: (i) obtaining clinical trial parameters from a plurality of historical clinical trials for a disease condition, said clinical trial parameters comprise investigator sites and trial enrollment rates, wherein said trail enrollment rate is defined as the number of patients enrolled in a unit of time; (ii) plotting a graph of investigator sites vs trial enrollment rates; and (iii) extrapolating from said graph a number of investigator sites corresponding to a desired trial enrollment rate, or a number of trial enrollment rate corresponding to a desired number of investigator sites, wherein said extrapolated number of investigator sites or number of trial enrollment rate would provide information for improving operational effectiveness of a clinical trial. In one embodiment, the investigator sites and trial enrollment rates are further converted into median investigator sites and median trial enrollment rates before plotting said graph.

In one embodiment, the unit of time is one month. In one embodiment, the disease condition is a metabolic disease condition, a respiratory disease condition, or a neurologic disease condition, and other disease conditions studied by randomized clinical trials.

In one embodiment, the graph of investigator sites vs trial enrollment rate can be fitted by the following formula:

CTER=A*(1−e^{BN})+C

wherein CTER is Clinical Trial Enrollment Rate; A, B, and C are parameters for a set of clinical trials of a disease condition; B is a negative constant for a set of clinical trials; and N is investigator sites. In one embodiment, the upper limit of trial level enrollment rate is A+C.

In another embodiment, the present invention provides a non-transitory computer-readable medium with instructions stored thereon, that when executed by a processor, perform the steps comprising: (i) obtaining from a first database clinical trial parameters from a plurality of historical clinical trials for a disease condition, said clinical trial parameters comprise investigator sites, trial enrollment rates, and site enrollment rates; (ii) plotting a graph of investigator sites vs trial enrollment rates, or a graph of investigator sites vs site enrollment rates; and (iii) extrapolating from a graph of investigator sites vs trial enrollment rates a number of investigator sites corresponding to a desired trial enrollment rate, or a number of trial enrollment rate corresponding to a desired number of investigator sites, or extrapolating from a graph of investigator sites vs site enrollment rates a number of investigator sites corresponding to a desired site enrollment rate, or a number of site enrollment rate corresponding to a desired number of investigator sites. In one embodiment, the investigator sites, trial enrollment rates or site enrollment rates are further converted into median investigator sites, median trial enrollment rates, or median site enrollment rates before plotting said graph.

In one embodiment, the above computer-readable medium further comprises instructions to perform a step of curve fitting after step (ii). In one embodiment, for a graph of investigator sites vs trial enrollment rates, the graph can be fitted by a formula:

CTER=A*(1−e^{BN})+C

wherein CTER is Clinical Trial Enrollment Rate; A, B, and C are parameters for a set of clinical trials of a disease condition; B is a negative constant for a set of clinical trials; and N is investigator sites.

In another embodiment, for a graph of investigator sites vs site enrollment rates, said graph can be fitted by a formula:

GSER=a*e^{bN}+c

wherein GSER is Gross Site Enrollment Rate; a, b, and c are parameters for a set of clinical trials of a disease condition; b is a negative constant for a set of clinical trials; and N is investigator sites.

In another embodiment, the present invention also provides a system for improving operational effectiveness of a clinical trial, comprising: (i) a memory for storing a database of clinical trial parameters derived from a plurality of historical clinical trials for a disease condition, said clinical trial parameters comprise investigator sites, trial enrollment rates, and site enrollment rates; and (ii) a processor that generates a graph of said investigator sites vs said trial enrollment rates, or a graph of said investigator sites vs said site enrollment rates, wherein said processor further extrapolating from said graph of investigator sites vs trial enrollment rates a number of investigator sites corresponding to a desired trial enrollment rate, or a number of trial enrollment rate corresponding to a desired number of investigator sites, or extrapolating from said graph of investigator sites vs site enrollment rates a number of investigator sites corresponding to a desired site enrollment rate, or a number of site enrollment rate corresponding to a desired number of investigator sites.

In one embodiment, this invention provides a method of designing a clinical trial enrollment plan, wherein said method comprises the steps of: (i) obtaining clinical trial parameters from a plurality of historical clinical trials for a disease condition, said clinical trial parameters comprise the number of investigator sites, site enrollment rates, and trial enrollment rates, wherein said site enrollment rate is defined as the number of patients enrolled at a single investigator site in a unit of time, said trial enrollment rate is defined as the number of patients enrolled in a unit of time; (ii) conducting non-linear regression analysis to model the relationship between the number of investigator sites and the site enrollment rates, or the relationship between the number of investigator sites and the trial enrollment rates; and (iii) extrapolating from said regression analysis one or more parameters selected from the group consisting of the number of investigator sites, site enrollment rates, and trial enrollment rates, wherein said extrapolated parameters are used in the design of one or more clinical trial enrollment plans. In one embodiment, the unit of time for the calculation of site enrollment rate or trial enrollment rate is one month.

In one embodiment, the above method of designing one or more clinical trial enrollment plans further comprises the step of calculating resources required for implementing each of said one or more clinical trial enrollment plans, wherein said resources comprise, for example, budget for treatment of said disease condition.

In one embodiment, said non-linear regression analysis comprises using GSER=a·e^{bN}+c, said GSER is Gross Site Enrollment Rate, e is an exponential function, a, b, c are constants to be determined in the non-linear regression, and N is the number of investigator sites. In another embodiment, c is the lower limit of the gross site enrollment rate. In one embodiment, said GSER is related to Site Effectiveness Index (SEI) and Average Site Enrollment Rate (ASER) as: GSER=SEI×ASER.

In one embodiment, said non-linear regression analysis comprises using CTER=A·(1−e^{BN})+C, said CTER is Clinical Trial Enrollment Rate, e is an exponential function, A, B, C are constants to be determined in the non-linear regression, and N is the number of investigator sites. In another embodiment, A+C is the upper limit of the clinical trial enrollment rate.

In one embodiment, the above disease condition is selected from the group consisting of a metabolic disease condition, a respiratory disease condition, a neurologic disease condition, and other disease conditions studied by randomized clinical trials.

In one embodiment, this invention further provides a non-transitory computer-readable medium with instructions stored thereon for designing a clinical trial enrollment plan, that when executed by a processor, perform the steps comprising: (i) obtaining from a first database clinical trial parameters from a plurality of historical clinical trials for a disease condition, said clinical trial parameters comprise the number of investigator sites, site enrollment rates, and trial enrollment rates, wherein said site enrollment rate is defined as the number of patients enrolled at a single investigator site in a unit of time, said trial enrollment rate is defined as the number of patients enrolled in a unit of time; (ii) conducting non-linear regression analysis to model the relationship between the number of investigator sites and the site enrollment rates, or the relationship between the number of investigator sites and the trial enrollment rates; and (iii) extrapolating from said regression analysis one or more parameters selected from the group consisting of the number of investigator sites, site enrollment rates, and trial enrollment rates, wherein said extrapolated parameters are used in the design of one or more clinical trial enrollment plans.

In one embodiment, said non-linear regression analysis comprises using GSER=a·e^{bN}+c, said GSER is Gross Site Enrollment Rate, e is an exponential function, a, b, c are constants to be determined in the non-linear regression, and N is the number of investigator sites. In another embodiment, c is the lower limit of the gross site enrollment rate. In one embodiment, said GSER is related to Site Effectiveness Index (SEI) and Average Site Enrollment Rate (ASER) as: GSER=SEI×ASER.

In one embodiment, said non-linear regression analysis comprises using CTER=A·(1−e^{BN})+C, said CTER is Clinical Trial Enrollment Rate, e is an exponential function, A, B, C are constants to be determined in the non-linear regression, and N is the number of investigator sites. In another embodiment, A+C is the upper limit of the clinical trial enrollment rate.

In one embodiment, said disease condition is selected from the group consisting of a metabolic disease condition, a respiratory disease condition, and a neurologic disease condition, or other disease conditions studied by randomized clinical trials.

In one embodiment, this invention further provides a system for designing a clinical trial enrollment plan, comprising: (i) a memory for storing a database of clinical trial parameters derived from a plurality of historical clinical trials for a disease condition, said clinical trial parameters comprise the number of investigator sites, site enrollment rates, and trial enrollment rates, wherein said site enrollment rate is defined as the number of patients enrolled at a single investigator site in a unit of time, said trial enrollment rate is defined as the number of patients enrolled in a unit of time; and (ii) one or more processors for conducting non-linear regression analysis to model the relationship between the number of investigator sites and the site enrollment rates, or the relationship between the number of investigator sites and the trial enrollment rates, wherein said one or more processors further extrapolate from said regression analysis one or more parameters selected from the group consisting of the number of investigator sites, site enrollment rates, and trial enrollment rates, wherein said extrapolated parameters are used in the design of one or more clinical trial enrollment plans.

The invention being generally described, will be more readily understood by reference to the following examples which are included merely for purposes of illustration of certain aspects and embodiments of the present invention, and are not intended to limit the invention.

### EXAMPLE 1

### Relationship Between Clinical Trial Enrollment Rate (CTER) and Investigator Sites

A sub-database of clinical trials meeting the following inclusion criteria was constructed: (i) interventional; (ii) with 10 or more sites; (iii) started in year 2000 or later; and (iv) completed enrollment at the time of analysis. The following trials were excluded: (i) extensional trials; (ii) registration trials; (iii) trials including healthy subjects; and (iv) trials with expanded access. Subsequently, a sub-database of relatively “homogeneous” clinical trials was constructed.

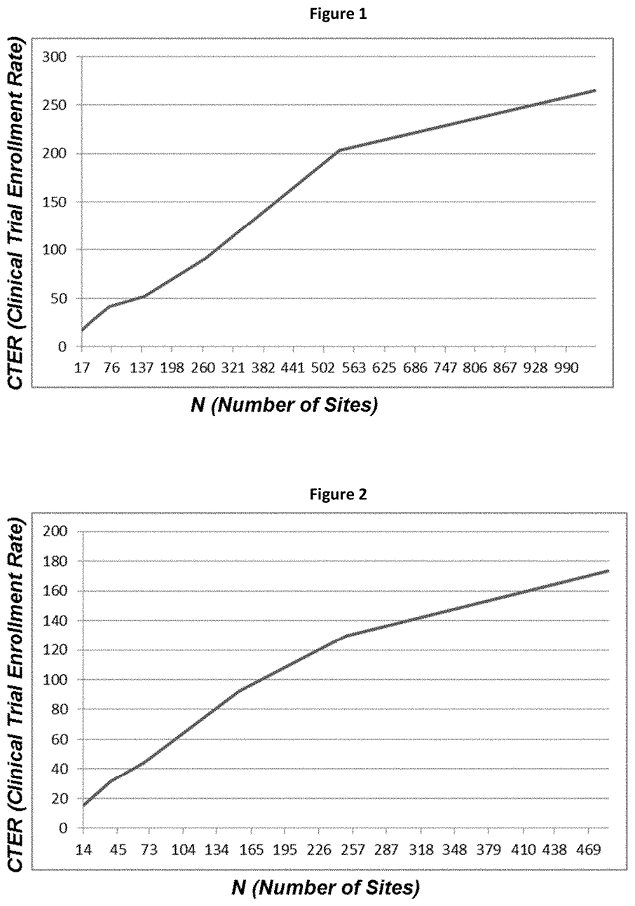

FIG. 1 shows a chart for clinical trials of a single metabolic disease condition. The following steps were taken to derive this chart:
Focus on trials with a single disease condition as primary condition;
Put the clinical trial into baskets according to number of sites:
   10 to 25 sites
   26 to 50 sites
   51 to 100 sites
   101 to 200 sites
   201 to 400 sites
   401 to 800 sites
   801 to more sites
Build a data table to pair median number of sites and median of trial level enrollment rate (CTER, Clinical Trial Enrollment Rate, number of patients enrolled per month) (Table 1);
Plot the data pairs in a chart.

TABLE 1

| Median Sites (N) | Median Trial Enrollment Rate (CTER) |
|---|---|
| 17 | 17.9 |
| 37 | 26.8 |
| 72 | 41.2 |
| 141 | 51.8 |
| 264 | 90.8 |
| 534 | 203 |
| 1047 | 265 |

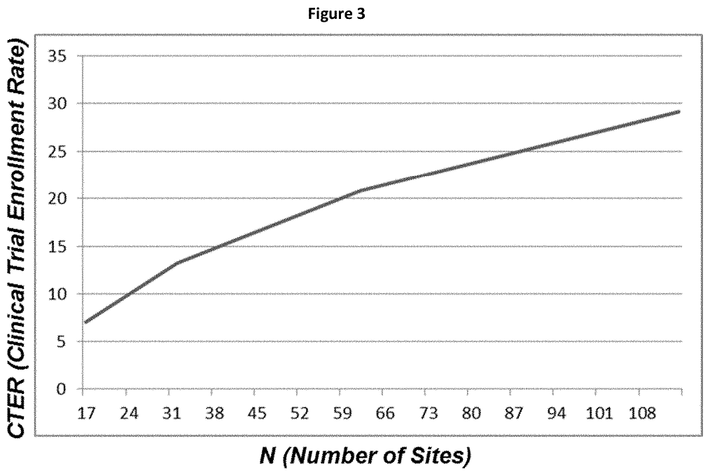

Following these same steps, charts for trials of a single respiratory disease conditions (FIG. 2) and trials of a single neurologic disease condition (FIG. 3) were constructed.

As a matter of fact, we can draw similar charts for group of clinical trials in every single disease conditions, when the sample size is big enough, and the disease condition is “pure” enough.

As vast majority of data points in the chart fall in narrow band, and in definable pattern, it is feasible to use this graph to forecast enrollment rate and other operational deliverables.

In each of the charts, there is a generalizable pattern: as more sites added to a clinical trial in the same disease condition, the enrollment rate at clinical trial level (CTER) increases. However, for every equal number of sites (N) added, the benefit to CTER diminishes. Eventually, the clinical trial level enrollment rate (CTER) will hit some sort of ceiling: the benefit from adding next batch of sites becomes negligible.

Thus, there is no “proportionate” relationship between number of sites and clinical trial enrollment rate (CTER). In other words, the relationship between sites and enrollment rate are not linear. When other factors equal, adding sites to a clinical trial CAN increase trial level enrollment rate, but at a diminished incremental benefit. Moreover, the benefit diminishes as more and more sites being added.

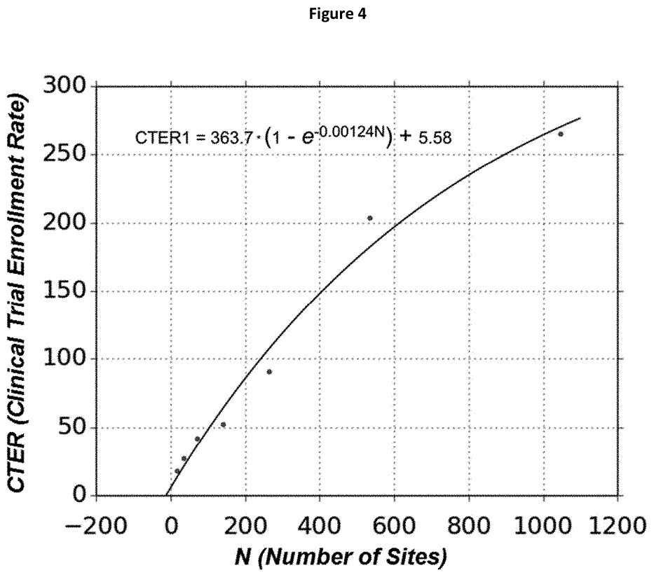

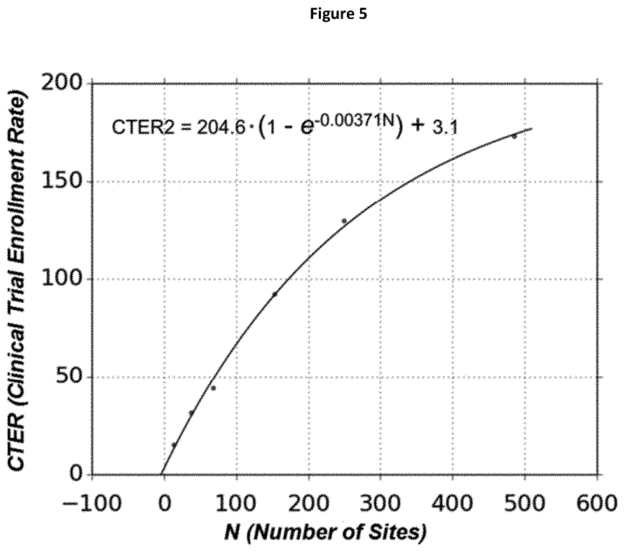

Each and every one of these charts seems to have distinct sizes and shapes. However, there is a simple equation to describe all the charts (see FIGS. 4-6). In one embodiment, the equation is:

CTER=A·(1−e^{BN})+C.

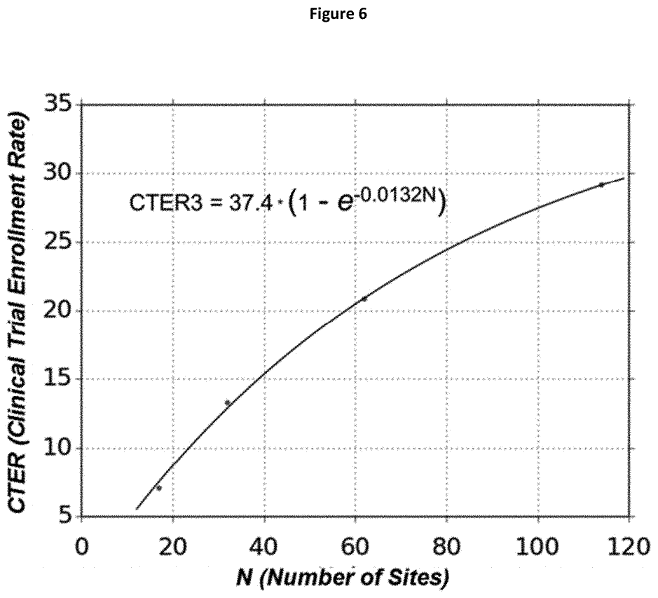

Let's use as an example the clinical trials shown in FIGS. 3 and 6:

CTER=37.4·(1−e^{-0.0132N}).

When CTER=10 patients per month is put into the chart, N=24. When CTER=20 patients per month is put into the chart, N=58. In another words, when other things are equal, if one want to double trial enrollment rate in order to shorten enrollment cycle time by half, one need to add more than twice as many sites to the pool (58 sites instead of 48 sites). Please note this is just an example to illustrate the concept. In reality, it is not usually possible to cut the enrollment cycle time by half.

By now, it is clear that there is an operational boundary for the planning and execution of clinical trials. When one keeps adding sites to a clinical trial, one will hit the ceiling at some point where there is no measurable benefit in gaining enrollment rate. Therefore it is safe to say that there is a limitation in terms of how far we can go to shorten enrollment cycle time by adding investigator sites.

The details of the operational boundaries are being discussed in mathematical terms below. In one embodiment, the following equation describes the relationship between CTER and clinical investigator sites (N):

CTER=A·(1−e^{BN})+C.

N is the number of clinical trial investigator sites; e is a mathematical constant as being defined by (1+1/n)ⁿ. It is approximately equal to 2.71828. B is a negative constant for a defined set of clinical trials (usually a single disease condition).

When N becomes infinitely big (when very large number of sites is used), e^{BN} become next to zero, and CTER will become close to A+C. In other words, no matter how many sites are being deployed in a clinical trial, it is not possible to exceed the trial level of A+C. In reality, one would like to get to as close as possible to A+C, by utilizing as few as possible sites (smaller N). A+C is the upper limit for trial level enrollment rate. Constants A, B, and C are parameters specific to a set of clinical trials of a specific and single disease condition.

Due to the non-linear nature of the enrollment rate and number of investigator sites, it is difficult to accurately obtain the constants a, b, c for GSER or A, B, C for CTER. In order to overcome this problem, non-linear regression is employed where data are modeled by a function which is nonlinear combination of the model parameters and are fitted by a method of successive approximations. The computations for non-linear regression analyses are not feasible without the use of a computer, and most statistical packages have routines for nonlinear regression. Further, statistical methods can be employed to obtain possible enrollment rates and number of investigator sites within a range of the regression model. A confidence belt can be obtained from the result of the nonlinear regression within which the enrollment rates and number of investigator sites are assumed to be achievable. For example, a 90% confidence belt. Segmented regression can also be used if it is believed that the relationship cannot be fitted in a single model.

### EXAMPLE 2

### Relationship Between Gross Site Enrollment Rate (GSER) and Investigator Sites

Using the same approach as discussed above for trial level enrollment rate (CTER), one can learn more about site level enrollment rate (GSER, Gross Site Enrollment Rate). Starting from the same sub-database as being used to understand CTER, the following steps were taken to build charts showing the relationship between number of sites (N) and site level enrollment rate (GSER):
Focus on trials with a single disease condition as primary condition;
Put the clinical trial into baskets according to number of sites:
   10 to 25 sites
   26 to 50 sites
   51 to 100 sites
   101 to 200 sites
   201 to 400 sites
   401 to 800 sites
   801 to more sites
Build a data table to pair median number of sites and median of site level enrollment rate (GSER, Gross Site Enrollment Rate, number of patients per site per month) (Table 2);
Plot the data pairs in a chart.

TABLE 2

| Median Sites (N) | Median Site Enrollment Rate (GSER) |
|---|---|
| 17 | 1.13 |
| 37 | 0.79 |
| 72 | 0.6 |
| 141.5 | 0.43 |
| 264.5 | 0.3 |
| 534 | 0.31 |
| 1047 | 0.29 |

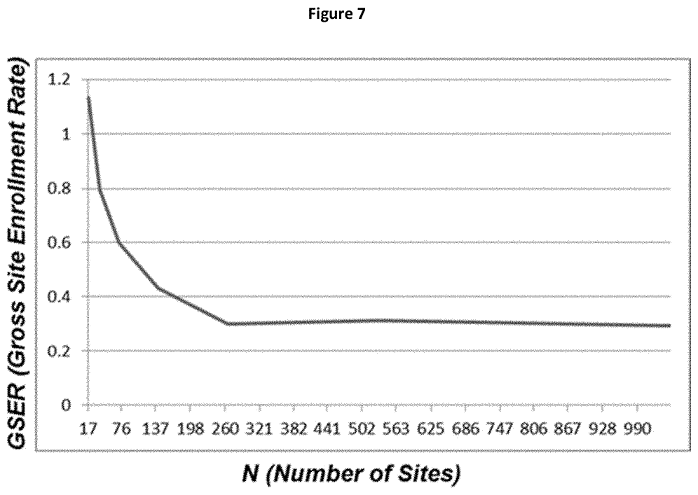

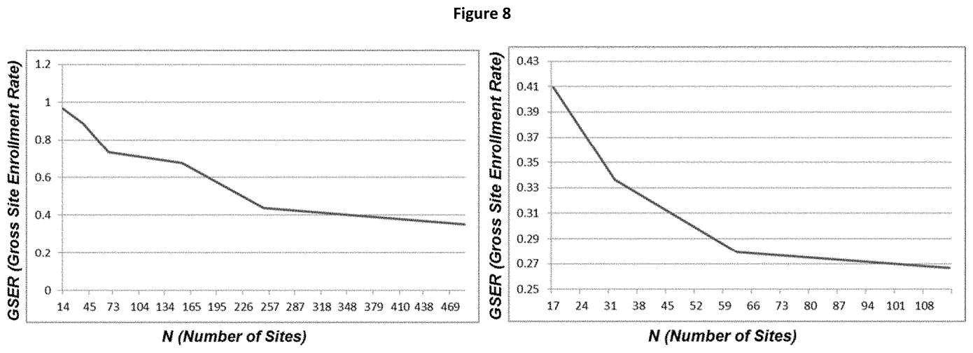

The charts shown in FIGS. 7-8 have different sizes and shapes. But the pattern is relatively simple: as the number of sites used in a set of clinical trials for a single disease condition increases, the site level enrollment rate (GSER) decreases. It is not a linear relationship. Rather, GSER drops much more quickly when the clinical trials involve smaller number of sites. It stabilizes at a certain level when the clinical trials become big enough.

As vast majority of data points in the chart fall in narrow band, and in definable pattern, it is feasible to use this graph to forecast enrollment rate and other operational deliverables.

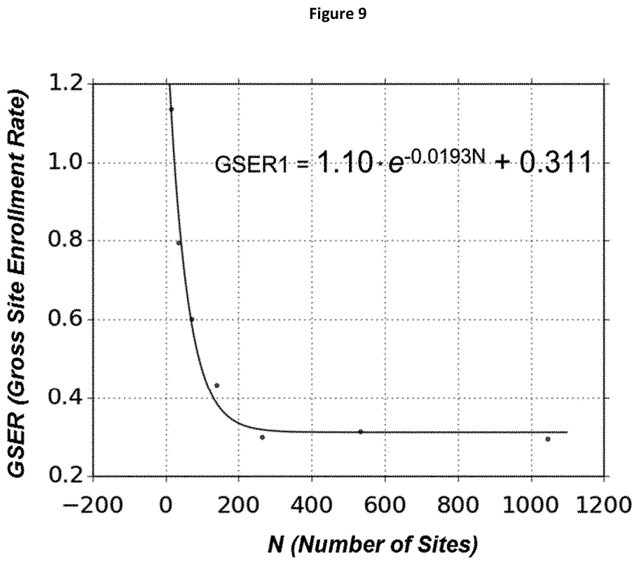

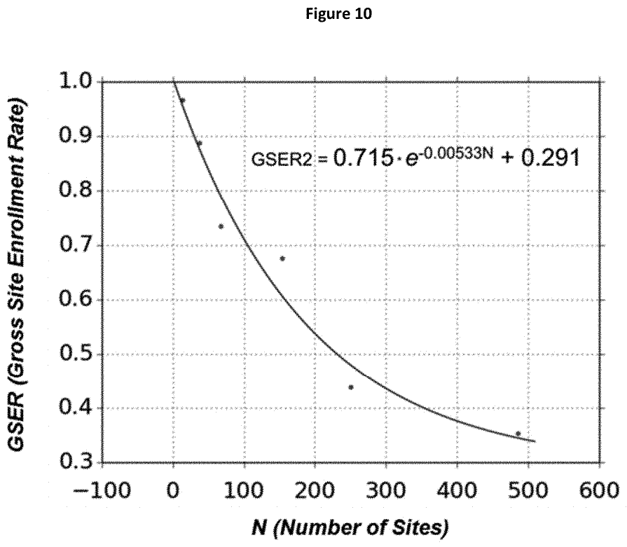

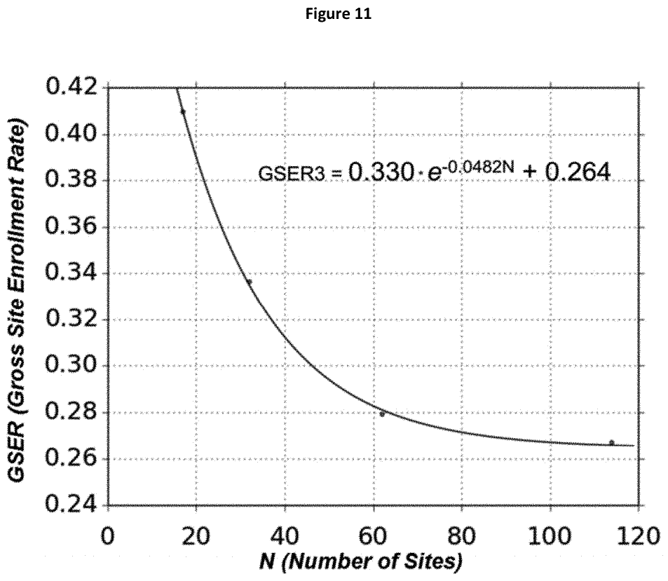

Again, there are mathematical relationships behind this pattern (see FIGS. 9-11). In one embodiment, the mathematical relationships can be represented by the following equation:

GSER=a·e^{bN}+c,

where b is a negative constant for a defined set of clinical trials (usually a single disease condition). When N becomes infinitely large (use of very large number of sites), e^{bN} becomes next to zero, and GSER will become close to c. That is to say, Gross Site Enrollment Rate cannot be smaller than c. The farther away one can stay from c by reducing the number of sites deployed in a clinical trial, the more one will be able to improve collective site enrollment performance in a clinical trial. In other word, c is the lower boundary for site level enrollment rate. Constants a, b, and c are parameters specific to a set of clinical trials of a specific and single disease condition.

As discussed above, when transitioning to a Phase III trial after a successful Phase II trial, one simply cannot apply the site enrollment rate in a usually smaller Phase II clinical trial to a usually much larger Phase III trial. The Gross Site Enrollment Rate (GSER) for a smaller Phase II trial, when other things equal, is larger than the GSER for a larger Phase III trial. When one try to extrapolate the operational results from a Phase II clinical trial to a larger Phase III clinical trial, and use the GSER to predict the enrollment cycle time for the planned larger Phase III trial, one end up with disappointing results: there would be longer enrollment cycle time, and frequently a “rescue mission” has to be launched.

Many factors can be used to help us understand why larger trials have lower site enrollment rates (GSER) than those of smaller trials. It has been established before that the enrollment performance for the pool of sites deployed in a clinical trial, as being measured by Average Site Enrollment Rate (ASER, number of patients per site per month), is impacted by the effectiveness of site activation process, which is measured by Site Effectiveness Index (SEI, 0%<SEI<100%). With the introduction of GSER, a simple formula can be used to link all of them together:

GSER=ASER×SEI.

Site Effectiveness Index (SEI) and Average Site Enrollment Rate (ASER) have been defined as (2, 3):

$$SEI = {\int }_{i = 1}^{N} ( E {t}_{i} - S {t}_{i} ) / [ ( {Et}_{s} - {St}_{s} ) \times N ] ,$$

Where Eti: The time (date) site i closed for patient enrollment; Sti: The time (date) site i opened for patient enrollment; N: maximum number of sites opened for enrollment in the duration of patient enrollment at the study level; Ets: The time (date) clinical study (trial) closed for patient enrollment; Sts: The time (date) clinical study (trial) opened for patient enrollment.

ASER=TE/∫ᵢ₌₁^{N}(Etᵢ−Stᵢ)

TE is Total Enrollment. When it is in the planning stage, TE is targeted patient enrollment. When historical data are being evaluated, TE is the actual number of patient enrolled in a clinical trial.

The above relationships have been tested and have provided superior site enrollment results consistently (4).

As more sites (N) are involved in a clinical trial, operational complexity increases, which will lead to the decrease of SEI, that in return, will reduce the site level enrollment rate (GSER). While it is always difficult to find high performing investigator sites, it becomes even more difficult when we need to identify even larger number of sites. It is not surprising that the average enrollment performance for a trial with larger number of sites will be lower than trials use smaller number of sites.

In one embodiment, the present invention would level the playground for stakeholders in clinical trial planning and execution by improving the effectiveness of communication among stakeholders, objectively rewarding colleagues to achieve quantifiable improvements, and provide actionable opportunities to improve operational deliverables through better site selection, better process, etc. The establishment of a reliable way to forecast enrollment rate, both at clinical trial level (CTER), and at site level (ASER), will greatly enhance our ability to achieve these objectives.

### EXAMPLE 3

### Determining Gross Site Enrollment Rate (GSER)

In planning an oncology clinical trial, a client proposes using 150 sites to enroll 189 patients in 21 months.

In order to determine whether the above parameters for the clinical trial are practical and feasible, clinical trial parameters such as enrollment cycle time, number of patients enrolled, and number of investigator sites used were first collected from clinical trials completed for the same or similar oncology indication. In one embodiment, clinical trials that enrolled between 100 and 300 patients were chosen to ensure that they possess similar operational complexity to the trial in planning. From these historical data, Gross Site Enrollment Rate (GSER) can be calculated with the following formula: GSER=number of patients enrolled/number of sites/enrollment cycle time.

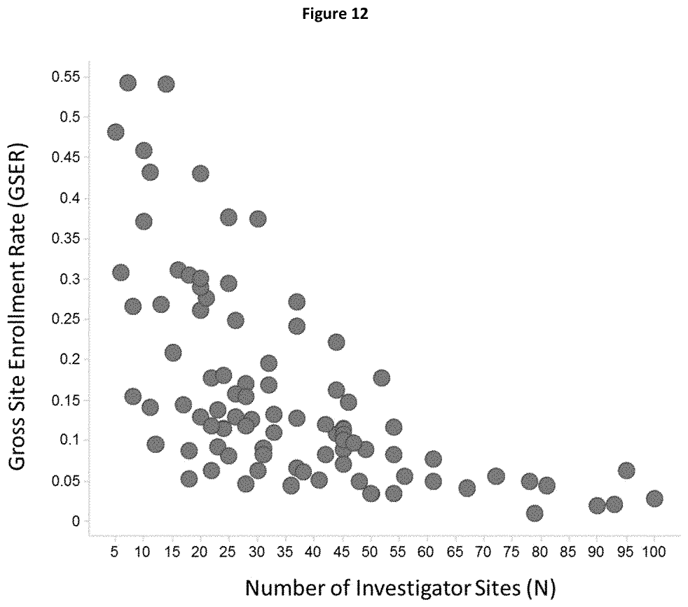

Next, a chart was plotted by plotting number of investigator sites (N) on the x axis, and GSER on the y axis (see FIG. 12). This chart depicts a clear pattern between the number of investigator sites and GSER, and one can use this chart to examine various scenarios for clinical trial planning. For example, in one embodiment, one may pick 45 as the number for investigator sites and determine from this chart the corresponding GSER for 45 investigator sites. With or without curve fitting, one may extrapolate from this chart that when N=45, it requires a corresponding or expected enrollment rate (GSER) of about 0.15 patients per site per month. This scenario falls inside the established pattern depicted by the chart, indicating that similar trials were successfully planned and executed before, and it is reasonable to expect that the planned trial can be successfully executed.

In another embodiment, one may pick 70 as the number for investigator sites and determine from this chart the corresponding GSER for 70 investigator sites. With or without curve fitting, one may extrapolate from this chart that when N=70, it requires a corresponding or expected enrollment rate (GSER) of about 0.12 patients per site per month. This scenario falls off the established pattern depicted by the chart. While there is no historical data to support these parameters (N=70 and GSER=0.12), since this scenario is very close to the established pattern, one may expect that a clinical trial using these two parameters would have a reasonably good chance to be successfully executed.

In yet another embodiment, one may determine from this chart the corresponding GSER for 150 investigator sites. As seen in the chart shown in FIG. 12, N=150 falls far away outside the established pattern depicted by the chart. Artificially extend the existing pattern shown in FIG. 12 would result in having enrollment rate (GSER) near zero, indicating a unreasonably long enrollment cycle time and it is not feasible to have a trail with N=150.

### REFERENCES

(1) Gen Li, Lauri Sirabella, 2010. Planning the Right Number of Investigative Sites for a Clinical Trial. The Monitor 2010; 24(4): 54-58.
(2) Gen Li. 2009. Finding the Sweet Spot. PharmExec Oct. 2, 2009.
(3) Gen Li, 2009. Site Effectiveness Index And Methods To Measure And Improve Operational Effectiveness In Clinical Trial Execution. U.S. Pat. No. 8,271,296
(4) Robert Gray, Gen Li, 2011. Performance-Based Site Selection Reduces Costs and Shortens Enrollment Time. The Monitor 2011; 25(7): 32-36.

What is claimed is:

1. A method of determining the number of investigator sites (N) for a new clinical trial associated with a disease using a computer, given a target enrollment time and a target number of patients, said method comprising:
(a) obtaining historical clinical trial data associated with said disease;
(b) deriving from said data values of Gross Site Enrollment Rate (GSER) defined as number of patients enrolled per site per unit of time;
(c) grouping said values of GSER into bins, each bin containing data associated with a distinct range of N values;
(d) determining the median value of N and median value of GSER in each bin, thereby obtaining multiple data points;
(e) establishing a quantitative relationship between N and GSER via non-linear regression by fitting said data points to the function GSER=a·e^{bN}+c, wherein a, b and c are constants and b is negative; and
(f) obtaining from said function a value of N, given said target enrollment time and target number of patients; wherein N is the number of investigator sites in said new clinical trial.

2. The method of claim 1, wherein said disease is selected from the group consisting of metabolic diseases, respiratory diseases, neurologic diseases and cancers.

3. The method of claim 1, wherein said unit of time is one month.

4. The method of claim 1, wherein the quantitative relationship between N and GSER is expressed as the formula GSER=1.10·e^{-0.0193N}+0.311 for clinical trials associated with a single metabolic disease.

5. The method of claim 4, wherein the unit of time is one month.

6. The method of claim 1, wherein the quantitative relationship between N and GSER is expressed as the formula GSER=0.715·e^{-0.00533N}+0.291 for clinical trials associated with a single respiratory disease.

7. The method of claim 6, wherein the unit of time is one month.

8. The method of claim 1, wherein the quantitative relationship between N and GSER is expressed as the formula GSER=0.330·e^{-0.00482N}+0.264 for clinical trials associated with a single neurologic disease.

9. The method of claim 8, wherein the unit of time is one month.

10. A method of determining the number of investigator sites (N) for a new clinical trial associated with a disease using a computer, given a target enrollment time and a target number of patients, said method comprising:
(a) obtaining historical clinical trial data associated with said disease;
(b) deriving from said data values of Gross Site Enrollment Rate (GSER) defined as number of patients enrolled per site per unit of time;
(c) grouping said values of GSER into bins, each bin containing data associated with a distinct range of N values;
(d) determining the median value of N and median value of GSER in each bin, thereby obtaining multiple data points;
(e) establishing a quantitative relationship between N and GSER via non-linear regression by fitting said data points to the function GSER=a·e^{bN}c, wherein a, b and c are constants and b is negative;
(f) obtaining from said function a value of N, given said target enrollment time and target number of patients; and
(g) enrolling said target number of patients at N investigator sites in said new clinical trial.
